(19) United States
(12) Patent Application Publication
(10) Pub. No.: US 2021/0145870 A1
(43) Pub. Date: May 20, 2021

(54) Silver Palladium and Silver Platinum Nanoparticles Useful as Antimicrobial and Anticancer Agents

(71) Applicant: Norheastern University, Boston, MA (US)

(72) Inventors: Aida Lopez RUIZ, Jamaica Plain, MA (US); Thomas J. WEBSTER, Barrington, RI (US)

(21) Appl. No.: 16/616,718

(22) PCT Filed: May 24, 2018
(86) PCT No.: PCT/US18/34499

§ 371 Date: Nov. 25, 2019

Related U.S. Application Data
(60) Provisional application No. 62/510,745, filed on May 24, 2017.

(51) Int. Cl. A61K 33/38; A61K 33/24; A61K 33/243; A61K 47/10; A61K 9/14; A01N 59/16; A61P 35/00; A61P 31/04; A61K 9/00

(52) U.S. Cl. CPC A61K 33/38; A61K 33/24; A61K 33/243; A61K 47/10; A61K 9/0024; A01N 59/16; A61P 35/00; A61P 31/04; A61K 9/146

(57) ABSTRACT

A method for treating bacterial infection or cancer is provided. The method includes administering to a subject in need thereof an effective amount of a composition containing nanoparticles containing Ag and either Pt or Pd.

### CROSS REFERENCE TO RELATED APPLICATIONS

This application claims the benefit under 35 U.S.C. § 119 of U.S. Provisional Patent Appl. No. 62/510,754, filed May 24, 2017, which is incorporated by reference herein in its entirety.

### BACKGROUND

Recent overuse of antibiotics has led to the growing prevalence of antibiotic-resistant bacteria.¹⁻³ The Centers for Disease Control and Prevention (CDC) expected at least 2 million people in the United States to become infected with drug-resistant bacteria in 2017, leading to over 23,000 deaths.⁴ The CDC has also predicted more deaths from microorganisms than all cancers combined by 2050. With hospital acquired infections becoming more prevalent, especially among patients being treated for cancer who become immunocompromised, it is of great concern that not many options exist to combat these infections.⁵

The use of antibiotics leads to selection for resistant bacteria.⁶ The generation of antibacterial resistance is mainly caused by the improper use of antibiotics.² In the United States, an estimated 23×10⁶ kg of antibiotics are used annually.⁷ Half of these antibiotics are given to animals, which are ingested by humans when they consume food, leading to antibiotic overuse and generation of resistant bacteria.⁸ Bacteria develop resistance to antibiotics when genetic mutations reduce or eliminate the effects of these antibiotics.⁹ Resistant bacteria can then multiply and take the place of others that were killed.¹⁰ In addition, the DNA that codes for antibiotic resistance can be easily transferred to other cells of the same type in the bacterial population, further increasing drug resistance.¹¹ Gram-negative bacteria are of particular concern because they are resistant to numerous antibiotics (antibiotics that contain a β-lactam ring, such as penicillin, ampicillin, and methicillin).^{2, 12} The emergence of multi-drug resistant, gram-negative bacteria has affected the practice of medicine in almost every field.⁷

Cancer is another disease that has an enormous worldwide impact. According to the National Cancer Institute, 1,685,210 new cases of cancer were diagnosed in the United States in 2016, and 595,690 people died from the disease. The use of chemotherapeutic drugs increases the risk of infections. Thus, there is a need for developing agents that have both antibacterial and anticancer effects.¹³

Nanotechnology is a multidisciplinary field with applications in many areas including the treatment of cancer.¹⁴ It has potential also as a tool in reducing infection.¹⁵ Use of nanomaterials made of silver, copper, selenium, palladium, and titanium dioxide has been reported for treating drug-resistant bacteria.¹⁶⁻¹⁷ Antimicrobial activity of nanoparticles is related in part to their small size and high surface area¹⁸, which allow nanoparticles to penetrate biofilms as well as bacterial cell walls, so that they can influence intracellular mechanisms. Nanoparticles have been used for treating cancer through incorporation of hydrophilic polymers that create a stealth surface for opsonization.¹⁴

### SUMMARY

In one aspect, the present technology provides a method for treating bacterial infection. The method comprises administering to a subject in need thereof an effective amount of a composition comprising a plurality of nanoparticles, the nanoparticles comprising an alloy of Ag and either Pt or Pd. The ratio of Ag to Pt or the ratio of At to Pd in the nanoparticles can be, for example 1:10, 1:5, 3:10, 2:5, 1:2, 3:5, 7:10, 4:5, 9:10, 1:1, 1.5:1, 2:1, 3:1, 4:1, 5:1, 6:1, 7:1, 8:1, 9:1, or 10:1. The nanoparticles can have a size (diameter) in the range of less than 100 nm, less than 80 nm, less than 70 nm, less than 60 nm, less than 50 nm, or less than 40 nm, or in the range from about 20 nm to about 100 nm, or from about 30 nm to about 70 nm, or from about 40 to about 60 nm, or from about 40 to about 50 nm. The administration of the nanoparticles preferably has a bacteriocidal or bacteriostatic effect in the subject. More preferably, the bacteriocidal or bacteriostatic effect is greater than that achieved by pure Ag nanoparticles by at least 20%, at least 30%, at least 40%, at least 50%, at least 100%, at least 2-fold, at least 3-fold, or at least 5-fold.

In another aspect, the present technology provides a method for treating cancer. The method comprises administering to a subject in need thereof an effective amount of a composition comprising a plurality of nanoparticles, the nanoparticles comprising an alloy of Ag and either Pt or Pd. The ratio of Ag to Pt or the ratio of At to Pd in the nanoparticles can be, for example 1:10, 1:5, 3:10, 2:5, 1:2, 3:5, 7:10, 4:5, 9:10, 1:1, 1.5:1, 2:1, 3:1, 4:1, 5:1, 6:1, 7:1, 8:1, 9:1, or 10:1. The nanoparticles can have a size (diameter) in the range of less than 100 nm, less than 80 nm, less than 70 nm, less than 60 nm, less than 50 nm, or less than 40 nm, or in the range from about 20 nm to about 100 nm, or from about 30 nm to about 70 nm, or from about 40 to about 60 nm, or from about 40 to about 50 nm. The administration of the nanoparticles preferably inhibits the growth and/or proliferation of cancer cells, or kills cancer cells, in the subject. More preferably, the anti-cancer growth, anti-cancer proliferation, or cancer cell killing effect is greater than that achieved by pure Ag nanoparticles by at least 20%, at least 30%, at least 40%, at least 50%, at least 100%, at least 2-fold, at least 3-fold, or at least 5-fold.

Another aspect of the technology is a medical device or consumer article comprising a plurality of nanoparticles on an exposed surface thereof, or impregnated within the article or device. The nanoparticles comprise or consist of an alloy of Ag and either Pt or Pd, and optionally comprise a nonionic surfactant. The medical device or consumer article can be, for example, a pump; an implantable defibrillator or portion thereof; an artificial organ; a surgical screw, tube, rod, pin, or mesh; a biodegradable device; an electrode; a surgical instrument; an eating implement, plate, cup, glass, or straw; a door knob or handle; an item of sports equipment; a shoe or boot liner; an item of clothing; a comb, brush, or razor; a toothbrush, toothpick, dental floss, or dental instrument or implant; a keyboard or computer tracking device; a touch screen of an electronic device; a remote control device; a musical instrument or part thereof; a writing implement; a packaging material for an article of commerce; or a button, control knob, steering wheel, or gear shift lever of a vehicle.

Yet another aspect of the technology is a composition comprising a plurality of nanoparticles. The nanoparticles comprise an alloy of Ag and either Pt or Pd, and the composition for use in preventing or reducing the growth of bacteria or cancer cells.

Still another aspect of the technology is the use of a composition comprising a plurality of nanoparticles to produce a medicament for preventing or reducing the growth of bacteria or cancer cells. The nanoparticles comprise an alloy of Ag and either Pt or Pd.

The technology is further summarized in the following listing of embodiments.

1. A method for treating bacterial infection, the method comprising administering to a subject in need thereof an effective amount of a composition comprising a plurality of nanoparticles, the nanoparticles comprising an alloy of Ag and either Pt or Pd.
2. The method of embodiment 1, wherein the nanoparticles further comprise a nonionic surfactant.
3. The method of embodiment 2, wherein the nanoparticles comprise or consist of Ag, Pd, and a nonionic surfactant.
4. The method of embodiment 2, wherein the nanoparticles comprise or consist of Ag, Pt, and a nonionic surfactant.
5. The method of any of embodiments 2-4, wherein the nonionic surfactant is polyethylene glycol hexadecyl ether.
6. The method of any of the previous embodiments, wherein the diameter of the nanoparticles is in the range of about 30 to about 60 nm.
7. The method of any of embodiments 1-5, wherein the average diameter of the nanoparticles is less than about 50 nm.
8. The method of any of the previous embodiments, wherein the administration of said composition has a bacteriocidal effect or a bacteriostatic effect in the subject.
9. The method of embodiment 8, wherein the bacteriocidal or bacteriostatic effect is operative against Gram-negative, Gram-positive, and/or multidrug resistant bacteria in the subject.
10. A method for treating cancer, the method comprising administering to a subject in need thereof an effective amount of a composition comprising a plurality of nanoparticles, the nanoparticles comprising an alloy of Ag and either Pt or Pd.
11. The method of embodiment 10, wherein the nanoparticles further comprise a nonionic surfactant.
12. The method of embodiment 11, wherein the nanoparticles comprise or consist of Ag, Pd, and a nonionic surfactant.
13. The method of embodiment 11, wherein the nanoparticles comprise or consist of Ag, Pt, and a nonionic surfactant.
14. The method of any of embodiments 11-13, wherein the nonionic surfactant is polyethylene glycol hexadecyl ether.
15. The method of any of embodiments 10-14, wherein the diameter of the nanoparticles is in the range of about 30 to about 60 nm.
16. The method of any of embodiments 10-14, wherein the average diameter of the nanoparticles is less than about 50 nm.
17. The method of any of embodiments 10-16, wherein the administration of said composition inhibits the growth and/or proliferation of cancer cells, or kills cancer cells, in the subject.
18. The method of embodiment 17, wherein the administration of said composition also has a bacteriostatic or bacteriocidal effect in the subject.
19. The method of any of embodiments 10-18, wherein administration of an anticancer therapeutic agent is reduced or eliminated in the subject as a result of the administration of said composition.
20. A medical device or consumer article comprising a plurality of nanoparticles on an exposed surface thereof, or impregnated within the article, the nanoparticles comprising an alloy of Ag and either Pt or Pd, and the nanoparticles optionally comprising a nonionic surfactant.
21. The medical device or consumer article of embodiment 20, wherein said nanoparticles form a coating on said exposed surface, and the coating has a bacteriocidal effect or a bacteriostatic effect.
22. The medical device or consumer article of embodiment 20 or 21, wherein the nonionic surfactant is polyethylene glycol hexadecyl ether.
23. The medical device or consumer article of any of embodiments 20-22 which is an implantable medical device.
24. The medical device or consumer article of embodiment 20 or 21 which is a consumer article, wherein the nanoparticles are present on a surface of the consumer article configured for contact with a user.
25. The medical device or consumer article of any of embodiments 20-24 which is selected from the group consisting of a catheter; a wound dressing; a joint replacement device or portion thereof; a pump; an implantable defibrillator or portion thereof; an artificial organ; a surgical screw, tube, rod, pin, or mesh; a biodegradable device; an electrode; a surgical instrument; an eating implement, plate, cup, glass, or straw; a door knob or handle; an item of sports equipment; a shoe or boot liner; an item of clothing; a comb, brush, or razor; a toothbrush, toothpick, dental floss, or dental instrument or implant; a keyboard or computer tracking device; a touch screen of an electronic device; a remote control device; a musical instrument or part thereof; a writing implement; a packaging material for an article of commerce; and a button, control knob, steering wheel, or gear shift lever of a vehicle.
26. A composition comprising a plurality of nanoparticles, the nanoparticles comprising an alloy of Ag and either Pt or Pd, the composition for use in preventing or reducing the growth of bacteria or cancer cells.
27. The composition of claim 26, wherein the nanoparticles are as described in any of claims 1-9.
28. Use of a composition comprising a plurality of nanoparticles, the nanoparticles comprising an alloy of Ag and either Pt or Pd, to produce a medicament for preventing or reducing the growth of bacteria or cancer cells.
29. The use of claim 28, wherein the nanoparticles are as described in any of claims 1-9.

### BRIEF DESCRIPTION OF THE DRAWINGS

FIG. 1A is a schematic diagram of the synthesis of AgPt or Ag/Pd nanoparticles. Ag and Pt or Pd ions are combined with a coating agent (BRIJ 58) and subsequently reduced with L-ascorbic acid to generate dendrimer nanoparticles. FIG. 1B is a transmission electron micrograph of AgPt nanoparticles produced according to the method shown in FIG. 1A. The particles were imaged at a voltage of 60 kV and 25000× magnification.

FIG. 2 is an EDX (Energy Dispersive X-ray Analysis) spectrum of AgPt nanoparticles. The peak at 2.01 indicates platinum while those between 2.8 and 3.35 indicate silver.

FIG. 3A shows growth curves of gram-negative bacteria P. aeruginosa grown in the presence of different concentrations of AgPt nanoparticles, measured over a 24-hour period. The topmost curve represents growth in the absence of nanoparticles. The remaining curves (viewed from the top to bottom and from the origin) correspond, respectively, to 10 μg/mL, 25 μg/mL, 75 μg/mL, and 50 μg/mL of AgPt nanoparticles. FIG. 3B shows the Gompertz model lag phase time (λ; light filled circles, curve increasing with concentration) and asymptotic absorbance (A; dark filled circles, decreasing with concentration) for the growth of P. aeruginosa at different AgPt nanoparticle concentrations. FIG. 3C shows growth curves of gram-negative bacteria P. aeruginosa grown in the presence of different concentrations of Ag nanoparticles, measured over a 24-hour period. The topmost curve represents growth in the absence of nanoparticles. The remaining curves (viewed from the top to bottom and from the origin) correspond, respectively, to 25 μg/mL, 75 μg/mL, and 50 μg/mL Ag nanoparticles. The curve corresponding to 10 μg/mL Ag nanoparticles overlaps partially with that for 25 μg/mL Ag nanoparticles. FIG. 3D shows Gompertz model lag phase time (λ; light filled circles, upper curve) and asymptotic absorbance (A; dark filled circles, lower curve) for the growth of P. aeruginosa at different Ag nanoparticle concentrations.

FIG. 3E shows growth curves of multi drug resistant E. coli (gram-negative) grown in the presence of different concentrations of AgPt nanoparticles, measured over a 24-hour period. The topmost curve represents growth in the absence of nanoparticles. The remaining curves (viewed from the top to bottom and from the origin) correspond, respectively, to 10 μg/mL, 25 μg/mL, 50 μg/mL, and 75 μg/mL AgPt nanoparticles, respectively. FIG. 3F shows Gompertz model lag phase time (λ; light filled circles, rising curve) and asymptotic absorbance (A; dark filled circles, falling curve) for the growth of multi drug resistant E. coli at different AgPt nanoparticle concentrations. FIG. 3G shows growth curves of gram-negative bacteria multi drug resistant E. coli grown in the presence of different concentrations of Ag nanoparticles, measured over a 24-hour period. The topmost curve represents growth in the absence of nanoparticles. The remaining curves (viewed from the top to bottom and from the origin) correspond, respectively, to 10 μg/mL, 25 μg/mL, 50 μg/mL, and 75 μg/mL Ag nanoparticles, respectively. FIG. 3H shows Gompertz model lag phase time (λ; light filled circles, initially rising curve) and asymptotic absorbance (A; dark filled circles, initially falling curve) for the growth of multi drug resistant E. coli at different Ag nanoparticle concentrations.

FIG. 3I shows growth curves of S. aureus (gram-positive) grown in the presence of different concentrations of AgPt nanoparticles, measured over a 24-hour period. The topmost curve represents growth in the absence of nanoparticles. The second and third curves (viewed from the top to bottom and from the origin) correspond, respectively, to 10 μg/mL, 25 μg/mL AgPt nanoparticles. The growth curves corresponding to 50 μg/mL, and 75 μg/mL AgPt nanoparticles overlap (see the bottom curve). FIG. 3J shows Gompertz model lag phase time (λ; light filled circles, rising curve) and asymptotic absorbance (A; dark filled circles, falling curve) for the growth of S. aureus at different AgPt nanoparticle concentrations. FIG. 3K shows growth curves of gram-negative bacteria S. aureus grown in the presence of different concentrations of Ag nanoparticles, measured over a 24-hour period. The topmost curve represents growth in the absence of nanoparticles. All growth curves (those corresponding to 10 μg/mL, 25 μg/mL, 50 μg/mL, and 75 μg/mL Ag nanoparticles) overlap. FIG. 3L shows Gompertz model lag phase time (λ; light filled circles, initially rising curve) and asymptotic absorbance (A; dark filled circles, initially falling curve) for the growth of S. aureus at different Ag nanoparticle concentrations.

FIGS. 4A, 4B, and 4C show results of viability analysis based on colony count for multi drug resistant E. coli (4A), P. aeruginosa (4B), and S. aureus (4C) treated with AgPt nanoparticles for 9 hours, compared with a control having no nanoparticles. p-value<0.05; data shown is mean+/−SEM; N=3.

FIGS. 5A-5I show results of live/dead assays carried out with P. aeruginosa, multi drug resistant E. coli, and S. aureus, treated with Ag and AgPt nanoparticles at 10 μg/mL or with control (no Nanoparticles): P. aeruginosa with control (5A), P. aeruginosa with Ag Nanoparticles (5B), P. aeruginosa with AgPt Nanoparticles (5C), multi drug resistant E. coli with control (5D), multi drug resistant E. coli with AgNanoparticles (5E), multi drug resistant E. coli with AgPt Nanoparticles (5F), S. aureus with control (5G), S. aureus treated with Ag Nanoparticles (5H), and S. aureus treated with AgPt Nanoparticles (5I). Magnification 10×

FIG. 6 shows viability profiles of human dermal fibroblasts treated with AgPt Nanoparticles for 1, 3 or 5 days (measured against no nanoparticle control for the corresponding day). p-value<0.05; data shown is mean+/−SEM; N=3.

FIGS. 7A and 7B show cell viability results for melanoma (7A) and glioblastoma (7B) cells treated with AgPt Nanoparticles for 1, 3 or 5 days (measured against no nanoparticle control for the corresponding day). p-value<0.05; data shown is mean+/−SEM; N=3.

FIGS. 8A and 8B show live/dead assays of melanoma cells treated, respectively, with AgPt nanoparticles (10 μg/mL) or no nanoparticles (control). FIGS. 8C and 8D show live/dead assays of glioblastoma cells treated, respectively, with AgPt nanoparticles (10 μg/mL) or no nanoparticles (control). Magnification 10×.

FIGS. 9A and 9B are TEM images of AgPd nanoparticles produced according to the method shown schematically in FIG. 1A with Pd substituting for Pt. The images in FIGS. 9A and 9B are at 4000× and 5000× magnification, respectively.

FIG. 10 is an EDX spectrum of AgPd nanoparticles. Peaks corresponding to Ag and Pd are marked.

FIGS. 11A and 11B show the results of antimicrobial assays testing the viability of P. aeruginosa in the absence (control) or presence of AgPd Nanoparticles at 100 μg/mL, 150 μg/mL, and 200 μg/mL. Bacterial plates are shown in FIG. 11A, and a graph for percent viability is shown in FIG. 11B.

FIG. 12A shows the results of antimicrobial assays of FIG. 11 in the form of colony counts. FIG. 12B shows an enlarged view of the 200 μg/mL condition from FIG. 11B.

FIG. 13 shows the results of assessment of viability of multi drug resistant E. Coli grown in the absence (control) or presence of AgPd Nanoparticles at 100 μg/mL, 150 μg/mL, and 200 μg/mL. Bacterial plates are shown.

FIG. 14A shows the results of antimicrobial assays of FIG. 13 in the form of colony counts, and FIG. 14B shows the results as percent viability.

FIG. 15A shows growth curves of P. aeruginosa in the absence (control) or in the presence of AgPd nanoparticles at 150 μg/mL (left) or 100 μg/mL (right). FIG. 15B shows the same in the absence (control) or in the presence of AgPd nanoparticles at 250 μg/mL (left) or 200 μg/mL (right).

FIG. 16A shows growth curves of S. aureus in the absence (control) or in the presence of AgPd nanoparticles at 150 μg/mL (left) or 100 μg/mL (right). FIG. 16B shows the same in the absence (control) or in the presence of AgPd nanoparticles at 250 μg/mL (left) or 200 μg/mL (right).

FIG. 17A shows growth curves of multi drug resistant E. Coli in the absence (control) or in the presence of AgPd nanoparticles at 150 μg/mL (left) or 100 μg/mL (right). FIG. 17B shows the same in the absence (control) or in the presence of AgPd nanoparticles at 250 μg/mL (left) or 200 μg/mL (right).

### DETAILED DESCRIPTION

The present technology involves the use of nanoparticles containing alloys of silver with platinum or palladium in treating bacterial infections and cancer. The present technology combines the beneficial features of silver and platinum to produce nanoparticles having unexpectedly superior anticancer and/or antibacterial properties compared to silver nanoparticles, platinum nanoparticles, or palladium nanoparticles.

The nanoparticles of the present technology can be synthesized by a chemical reduction of a combination of silver ions and platinum and/or palladium ions. A detergent, such as BRIJ58 (polyethylene glycol hexadecyl ether), can be used in the preparation of the nanoparticles. Silver platinum alloy nanoparticles and silver-palladium nanoparticles of approximately 40 nm diameter were obtained. The small size of the nanoparticles is believed to allow better interaction between the metal nanoparticles and cellular components, thereby contributing to enhanced toxicity of the nanoparticles with respect to bacterial cells and cancer cells.

The Ag/Pt and Ag/Pd nanoparticles of the present technology are demonstrated herein to have the capability to kill not only gram-positive (e.g., S. aureus) and gram-negative bacteria (e.g., P. aeruginosa), but also to kill multidrug-resistant bacteria, such as multidrug-resistant forms of E. coli. This was shown using several different types of assays for antibacterial activity, including growth curve analysis, colony counting, and “live/dead” assays using a vital stain (e.g., the LIVE/DEAD bacterial viability kit of ThermoFisher). For example, superior results were obtained for the Ag/Pt and Ag/Pd nanoparticles compared to Ag nanoparticles for colony counting studies against each of the three-different kinds of bacteria tested. The Ag/Pt and Ag/Pd nanoparticles demonstrated a remarkable improvement in antibacterial activity compared to the effect of nanoparticles containing silver alone. The use of pure silver nanoparticles, or nanoparticles comprising silver but lacking in platinum and palladium, is excluded from the present technology. Further, results described herein indicate that the Ag/Pt and Ag/Pd nanoparticles not only can prevent bacterial growth, but that they can also kill bacteria, even multidrug-resistant bacteria.

The capability of the Ag/Pt and Ag/Pd nanoparticles to kill cancer cells was also tested. Death of melanoma and glioblastoma cells was demonstrated with cell proliferation assays as well as with cell viability assays. Further, cytotoxicity assays carried out with human dermal fibroblast cells (non-cancerous cells) showed absence of significant toxicity when the nanoparticles were used at 10, 25, and 50 μg/mL concentrations. Nanoparticles containing an alloy of Ag and Pt showed lower toxicity against fibroblasts than against cancer cells or bacterial cells.

Examples below describe the studies carried out for examining the antibacterial and anticancer properties of the nanoparticle composites of the present technology in greater detail.

### EXAMPLES

### Example 1. Preparation and Characterization of Nanoparticles

Nanoparticles containing Ag/Pt and Ag/Pd were prepared according to a method adapted from Shim et al.²⁶ The morphology, structure, and size of the nanoparticles were characterized using transmission electron microscopy (TEM) (JEM 1010, JEOL) at 80 kV acceleration. For the composition of the nanoparticles, scanning electron microscopy (SEM) with energy dispersive X-ray analysis (EDX) was used with a Hitachi S-4800 scanning electron microscope equipped for EDX. The charge of the nanoparticles was measured by using a NanoBrook 90Plus particle size and zeta potential analyzer (Brookhaven Instruments, Holtsville, N.Y.). For TEM and EDX samples, nanoparticles were dried on 300-mesh carbon-coated copper grids (Electron Microscopy Sciences).

For the synthesis of silver palladium nanoparticles, 3 mL of each of K₂PdCl₄ (5 mM) and AgNO₃ (5 mM) (aqueous solution) were mixed in a vessel with sonication for 5 minutes. Then, 1 mL of 44 mM Brij 58 solution was added and sonication continued for another 5 minutes. Finally, 3 mL of 0.1 M L-ascorbic acid solution was added, and the reaction was allowed to proceed at room temperature for 45 minutes with sonication. After completion of the reaction, a precipitate was collected by centrifugation (10,000 rpm for 10 minutes) and washed with DI water to remove the excess surfactant and excess reactants. When the metal ions were reduced, the solution turned black, and nanoparticles were formed.

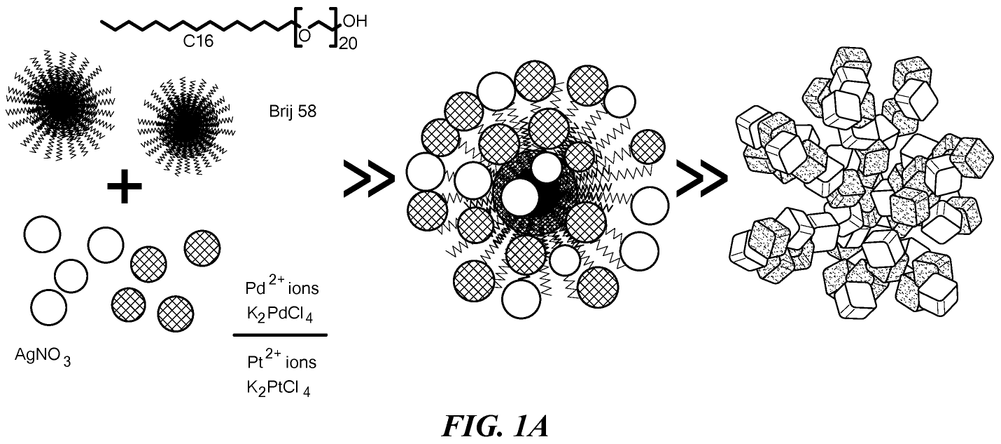

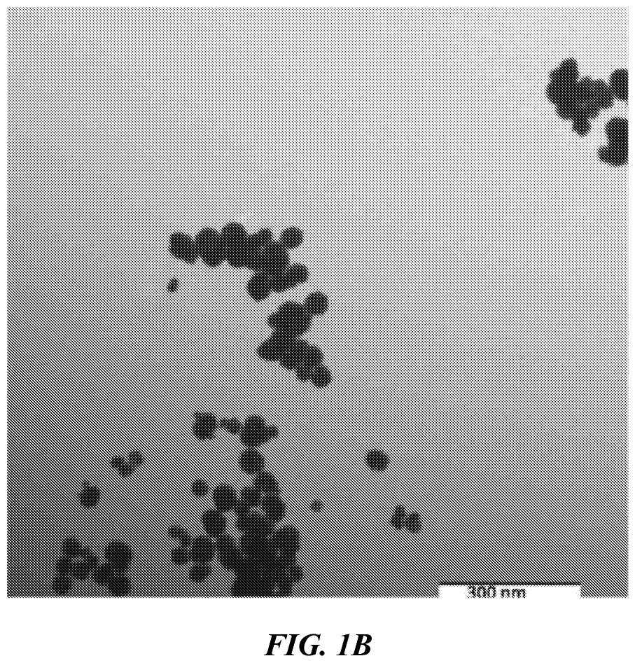

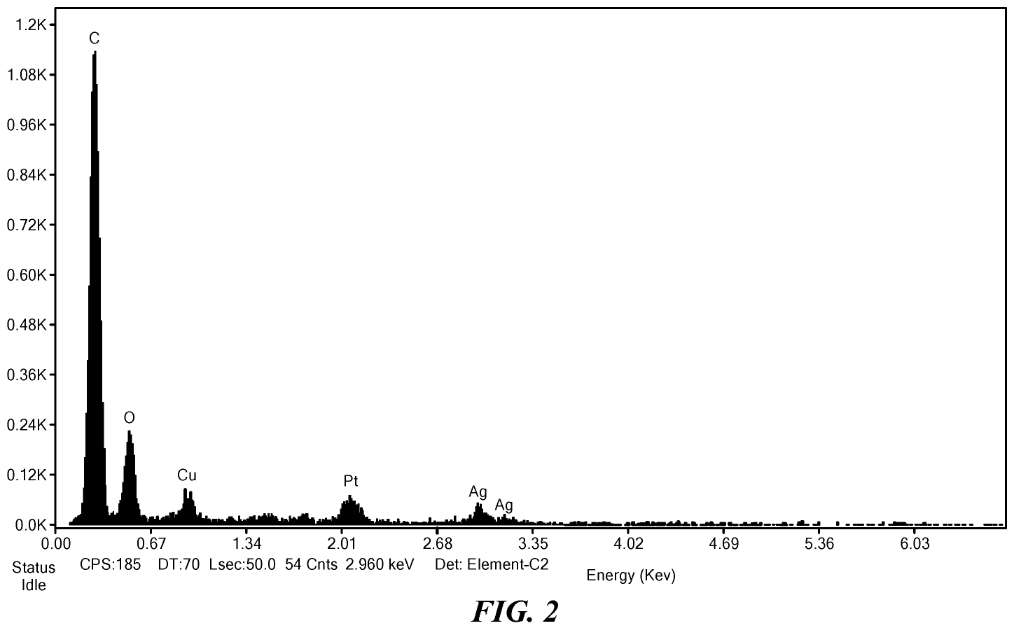

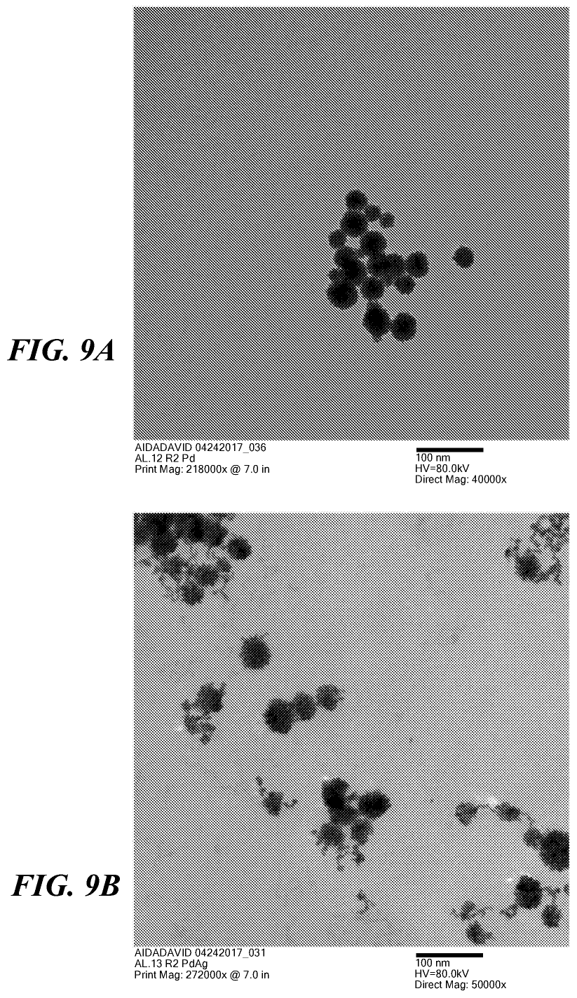

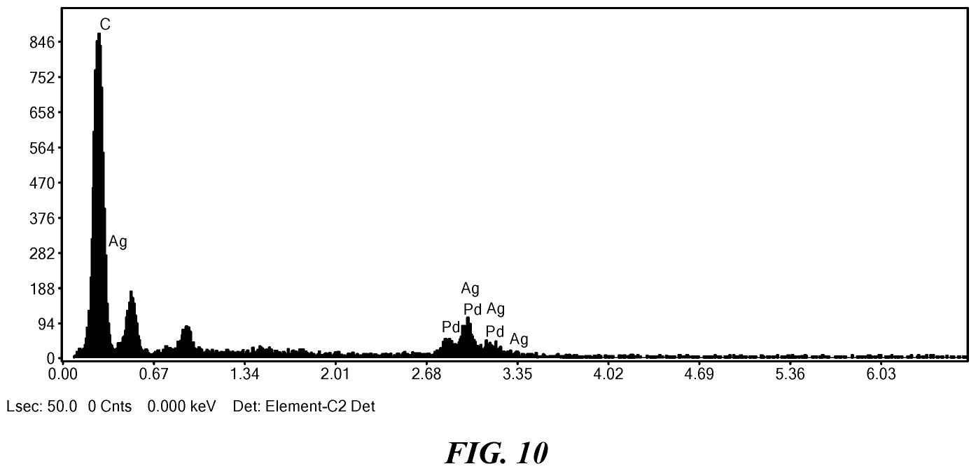

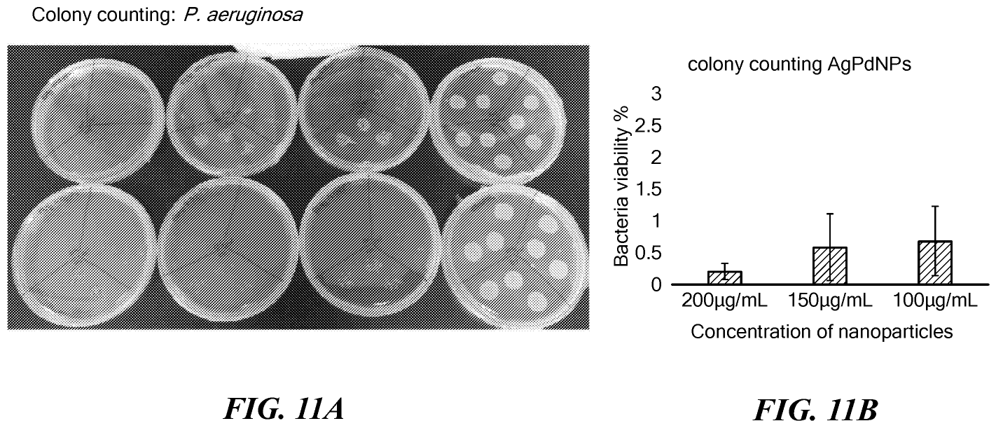

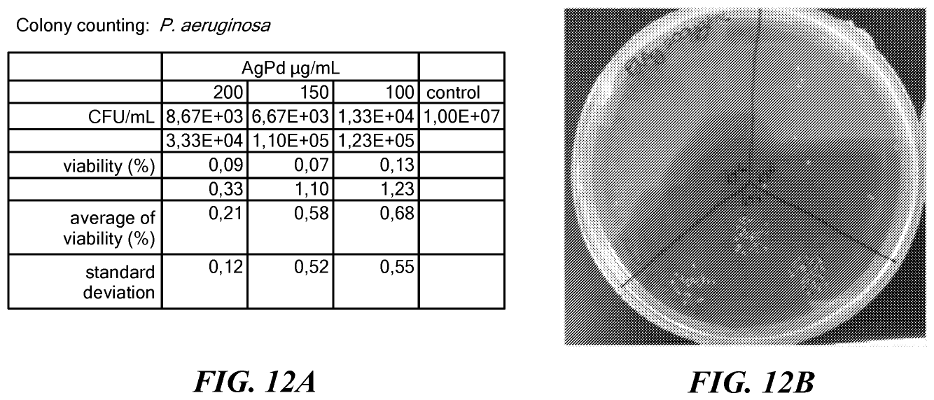

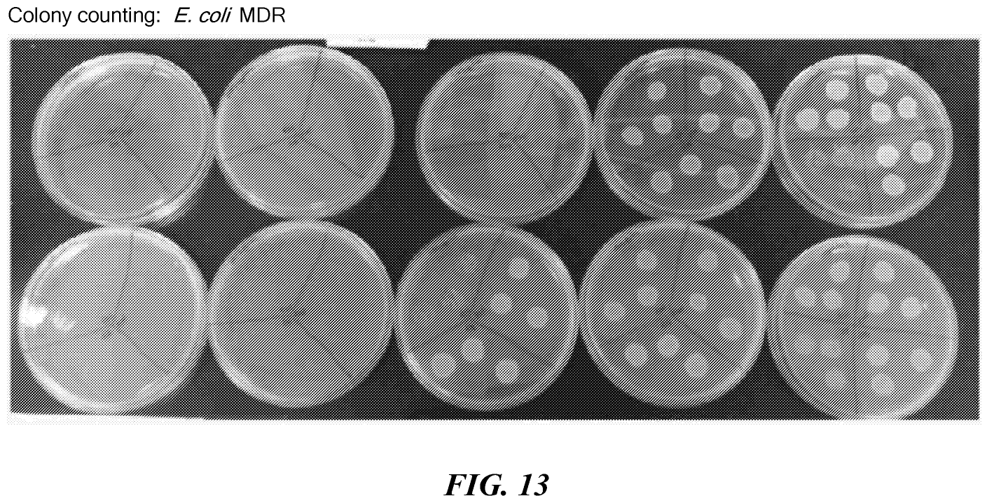

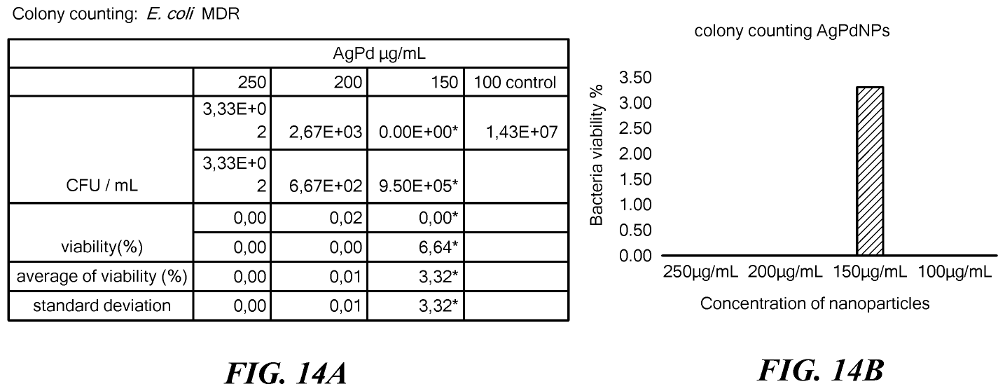

Analysis showed the nanoparticles to have a spherical shape with a surface covered with small blebs, (see TEM images of FIG. 1B, AgPt nanoparticles, and FIGS. 9A and 9B, AgPd nanoparticles). The diameter of the AgPt nanoparticles ranged from about 20 to about 60 nm, which an average diameter of 42.5±11 nm (mean±standard deviation). The nanoparticles had a zeta potential of −30 mV, indicating a negative surface charge and a reasonably stable structure; however, the nanoparticles had a slight tendency to aggregate, as visible in the TEM images. The composition of the nanoparticles was confirmed by EDX (FIGS. 2 and 10). The peak at 2.1 keV and the peaks between 2.8 and 3.5 in the EDX spectrum (FIG. 2) indicated the presence of Pt or Pd, respectively. Peaks from Ag, C and O from BRIJ58, and Cu from the grid also were detected.

### Example 2. Antibacterial Assays

Bacterial growth curve: To test the antibacterial effects of nanoparticles, different concentrations of the nanoparticles (Ag, AgPd, or AgPt) were added to 100 μL portions of a bacterial culture in the wells of a 96-well plate. The bacterial culture was prepared by incubating a single bacterial colony with tryptic soy broth (TSB) medium for 18 hours at 37° C. in a shaking incubator. At this point, the concentration of the bacteria was determined by measuring absorbance at 600 nm (OD₆₀₀). Next, the bacterial culture was diluted to obtain a final bacterial cell density of 2×10⁵ CFU/mL. The 96-well plate was incubated at 37° C. inside a spectrophotometer (SpectraMax Paradigm, Molecular Devices). OD₆₀₀ was measured every 2 minutes for 24 hours. All experiments were repeated three times. Bacterial growth curves were generated for each of P. aeruginosa (Gram-negative), S. aureus (Gram positive), and multi-drug-resistant E. coli (Gram negative).

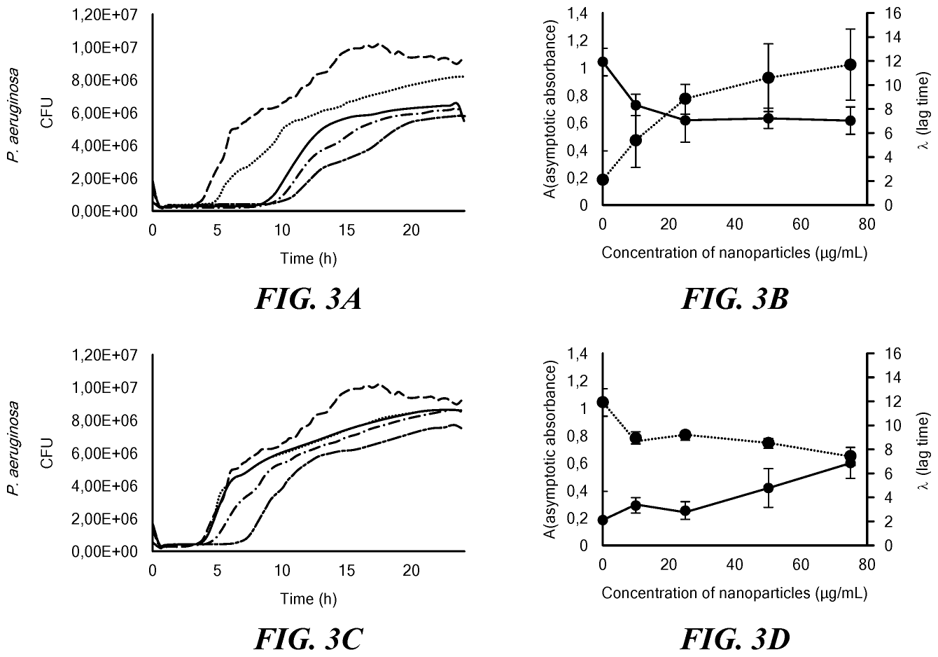

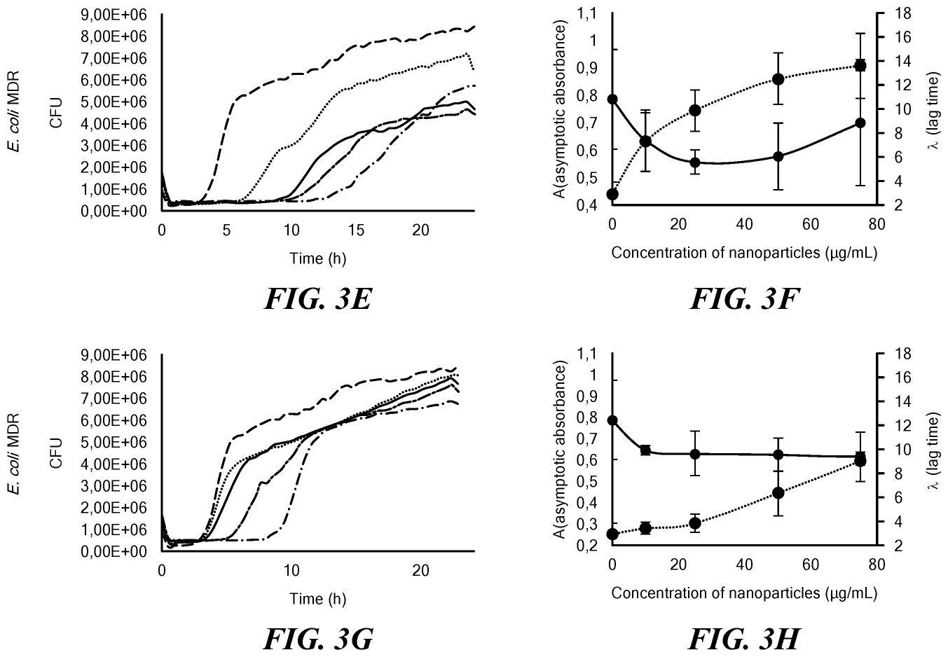

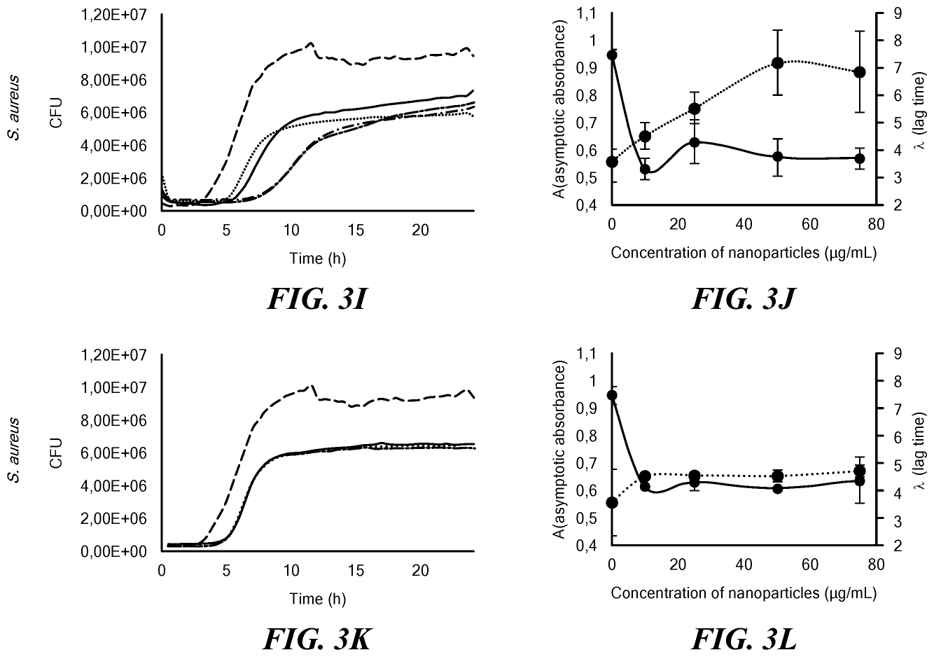

FIGS. 3A-3L show results for the effects of two different types of nanoparticles (AgPt and Ag), at concentrations of 10, 20, 50, and 75 μg/mL, on the growth of P. aeruginosa, S. aureus, and multi-drug-resistant E. coli. Both Ag and AgPt nanoparticles inhibited the growth of all three types bacteria (FIGS. 3A-3L). Compared to incubation with Ag nanoparticles, AgPt nanoparticles demonstrated significantly greater antibacterial effect. As the concentration of nanoparticles increased, the bacterial growth activity decreased. The antibacterial effect was lower for Gram-positive bacteria than for Gram-negative bacteria. This may be due to the thicker peptidoglycan cell wall of Gram-positive bacteria which could be more resistant to the uptake of the nanoparticles.

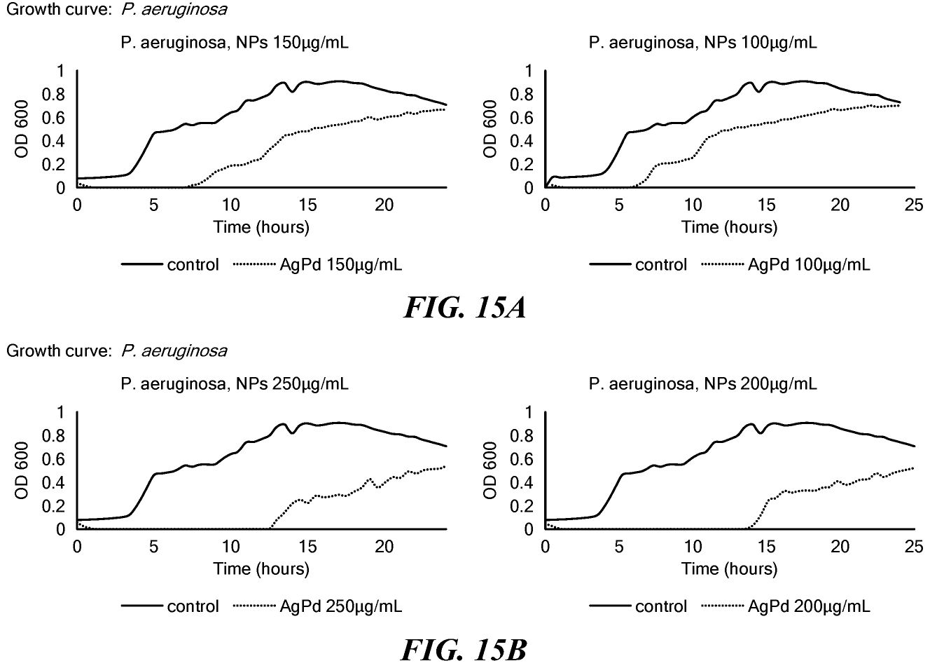

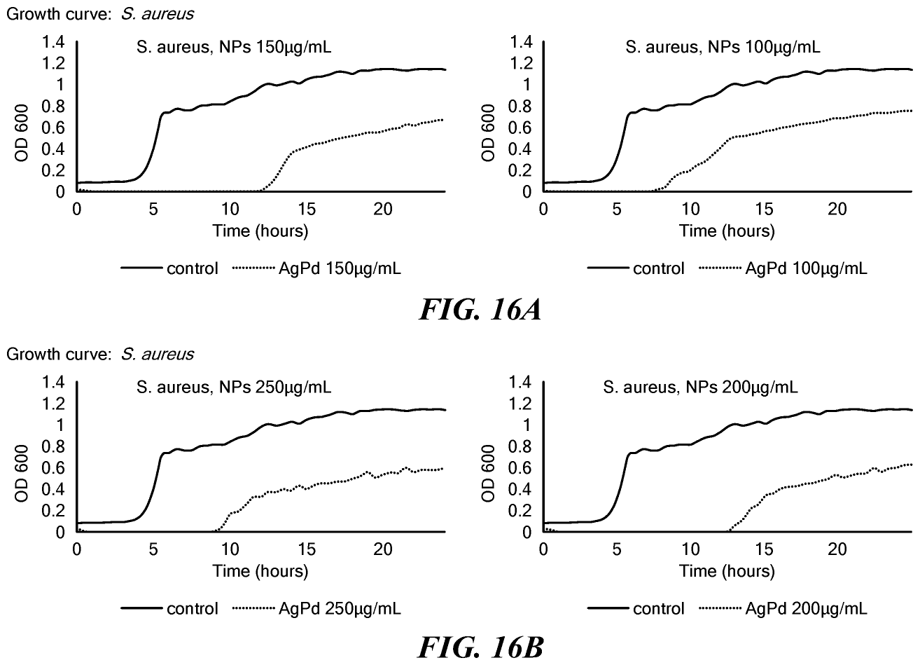

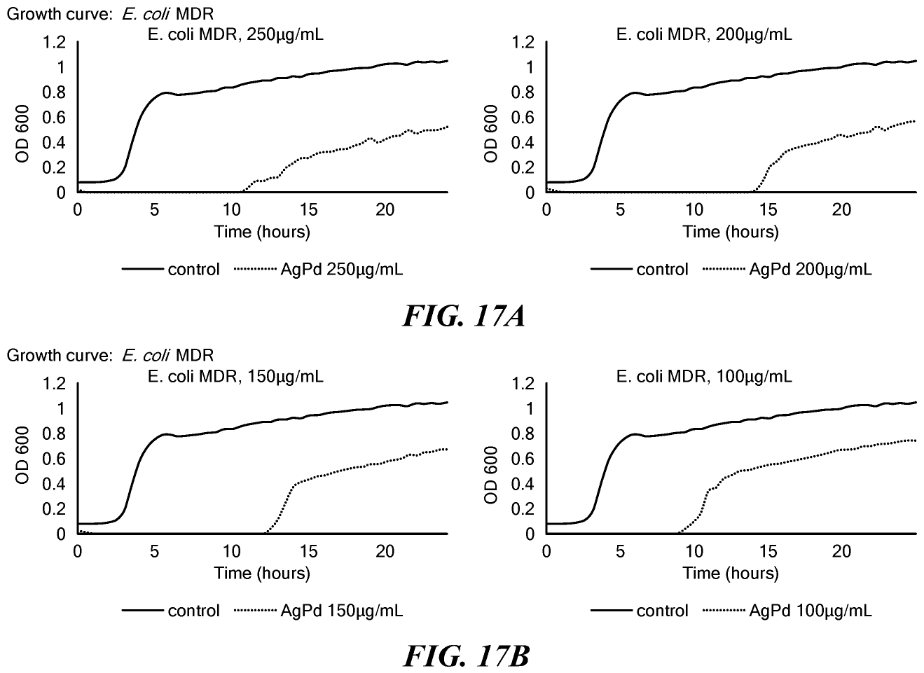

AgPd nanoparticles also inhibited the growth of each type of bacteria. See FIGS. 15A, 15B, 16A, 16B, 17A and 17B. AgPd nanoparticles were used at concentrations of 100, 150, 200, and 250 μg/mL.

To obtain growth curves from optical density measurements of bacterial density, a modified Gompertz model was used:

$$y = A exp \{ - exp [ \frac{{μ}_{m} e}{A} ( λ - t ) + 1 ] \}$$

where A is the asymptotic absorbance, μₘ is the maximum exponential growth rate, λ is the lag time, t is the time, and y is the absorbance.

According to the Gompertz model curve fitting results, the calculated lag time became significantly higher as the concentration of nanoparticles increased. However, the maximum asymptotic absorbance showed the reverse trend. With higher concentrations of nanoparticles, lower A was obtained. With this value, it was demonstrated that the use of AgPt nanoparticles affected bacterial growth significantly for Gram-positive, Gram-negative, and multidrug resistant bacteria.

Colony counting. Nanoparticles at 10 μg/mL concentration were added to wells of a 96-well plate containing 100 μL of a bacterial suspension each. The plate was incubated at 37° C. for 8 hours to allow the bacteria to reach the exponential growth phase. To prepare the agar plates, different dilutions of bacteria were prepared and once the agar plate was labeled with the concentration of bacteria in every region, 30 μL of the bacterial suspension was introduced into each region. Then, the plates were incubated for 9 hours at 37° C. and colony unit formation measured. All the experiments were repeated three times.

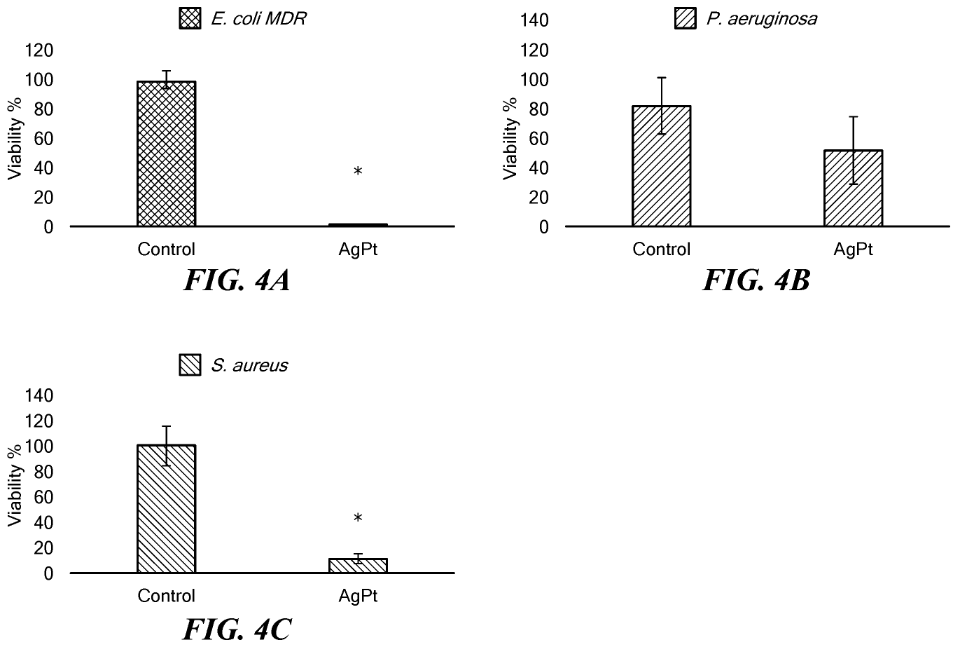

In agreement with the results obtained with growth curve assays, colony counting experiments (FIGS. 4A-4C) showed that AgPt nanoparticles had a strong antimicrobial effect even at the minimum concentration used (10 μg/mL). For P. aeruginosa, incubation with AgPt nanoparticles resulted in lowering of viability to 60% compared to incubation with no nanoparticles. For multi-drug resistant E. coli, viability observed was 1%, and for S. aureus, it was 12% relative to incubation with no nanoparticles. These results show that AgPt nanoparticles are effective as antimicrobial agent.

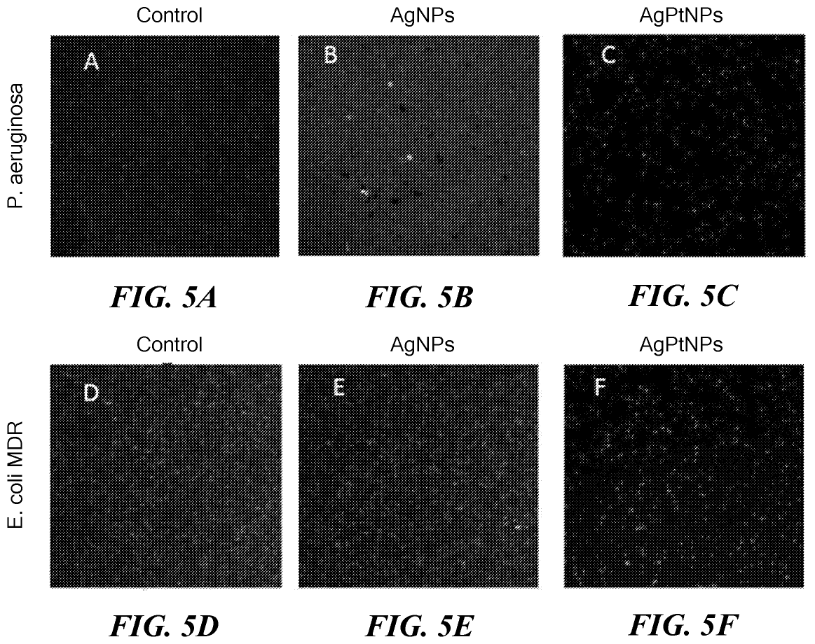

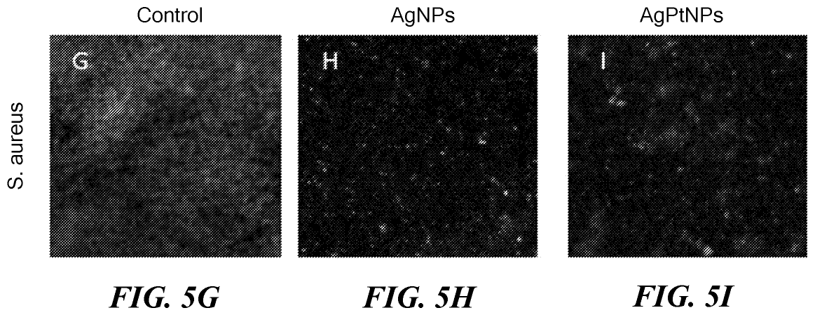

Cell Viability Assays. A LIVE/DEAD bacterial assay was performed using fluorescence microscopy to obtain a qualitative measurement of the effect of nanoparticles on bacteria. Each type of bacteria (1×10⁶ CFU/mL) was incubated in a 96-well plate with AgPt and Ag nanoparticles for 24 hours. Next, the plate was centrifuged to remove the supernatant and 100 μl of 0.85% NaCl was added (two times). Immediately thereafter, 1.25 μL of each fluorescent solution from the BacLight™ bacterial viability kit (ThermoFisher Scientific) in 1 mL of 0.85% NaCl solution was added to each well and kept in the dark for 15 minutes. Finally, the solution was centrifuged, washed with 0.85% NaCl, and resuspended for fluorescence microscopy (Zeiss inverted fluorescence microscope SpectraMax). Results (FIGS. 5A-5I) showed an increased number of spots (red spots) corresponding to dead bacteria in the presence of nanoparticles, thereby confirming results obtained from growth curve and colony counting measurements. Not only did the nanoparticles kill bacteria, they also did not allow living bacteria to grow, indicating that the nanoparticles are bacteriostatic, i.e., they stop bacteria from reproducing without necessarily killing all of them. Also, it is clear from the results that AgPt nanoparticles are more effective than Ag nanoparticles alone.

### Example 3. Anticancer Assays

Cytotoxicity Assays. To measure the toxicity of nanoparticles to healthy human cells, MTS assays were performed on human dermal fibroblasts (HDF) (Detroit 551-CCL-110; ATCC) cultured with and without nanoparticles. The purpose of using HDF was to examine if the toxicity of nanoparticles was low enough that that they might be used as control cells for determining the effectiveness of AgPt nanoparticles on cancer cells. The cells were cultured in Dulbecco's Modified Eagle's Medium (DMEM), complemented with 10% fetal bovine serum (FBS) and 1% penicillin-streptomycin in 75 mL flasks. Upon reaching 80% confluence, the cells were detached using trypsin (0.05% for 5 min), collected by centrifugation at 1100 rpm for 5 min, and used in CELLTITER 96 AQueous One Solution Cell Proliferation assays (MTS). For the assays, cells were cultured in a 96-well plate at a concentration of 50,000 cells/mL and incubated for 24 hours at 37° C. The medium was replaced with different concentrations of nanoparticles (10, 25, 50, 75, 100, 150, 200, and 250 μg/mL) in cell culture medium and incubated for 1, 3, or 5 days. To determine viability, the medium was removed, MTS solution added, and incubation continued for 3 hours at 37° C. The absorbance of the wells was measured at 490 nm using a spectrophotometer (SpectraMax Paradigm, Molecular Devices). All experiments were performed three times.

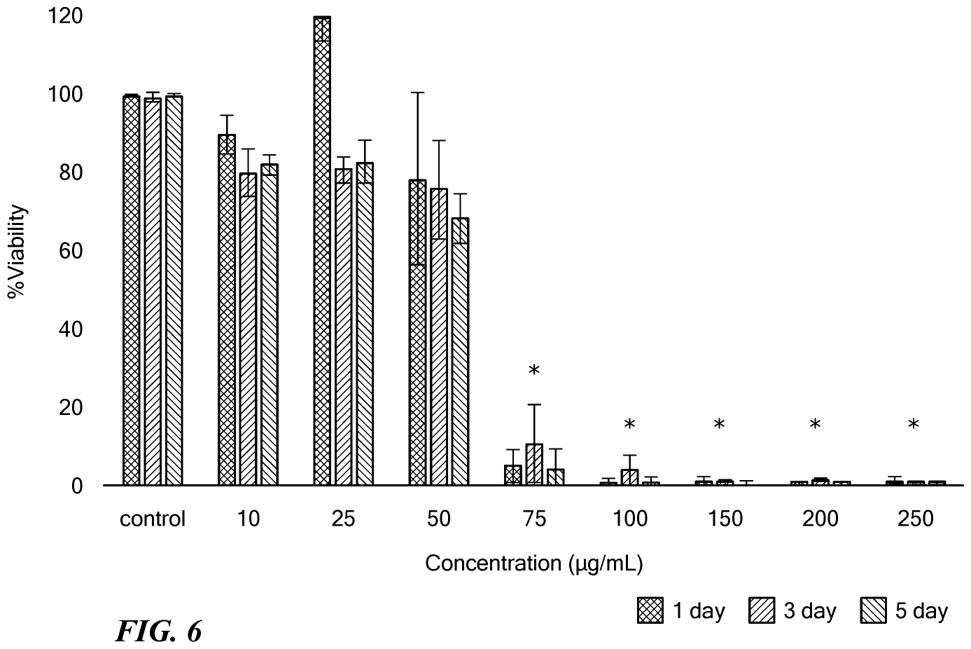

The results obtained are shown in FIG. 6. It was observed that at concentrations less than 75 μg/mL, a high percentage of HDF were viable. Moreover, the differences in viability between cells treated with 10, 25, and 50 μg/mL nanoparticles were not significant, indicating that nanoparticles are toxic for fibroblasts only at high concentrations (75 μg/mL or above). Additionally, viability of cells at 1, 3, and 5 days were similar, implying that the period of time during which the cells were cultured with the nanoparticles did not influence toxicity; toxicity of the nanoparticles was thus quickly acting. The IC₅₀ obtained for AgPt nanoparticles was 100 μg/mL for all days measured.

A comparison of the effect of AgPt nanoparticles on bacteria and fibroblast cells showed that nanoparticles may be used at 10, 20, and 50 μg/mL for the antibacterial effect without concern for cytotoxicity to mammalian host cells.

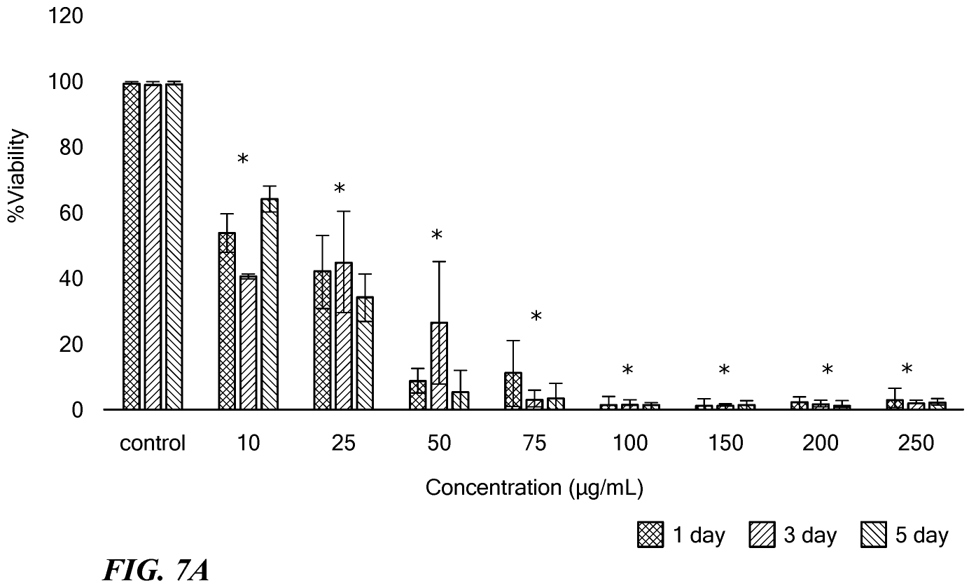

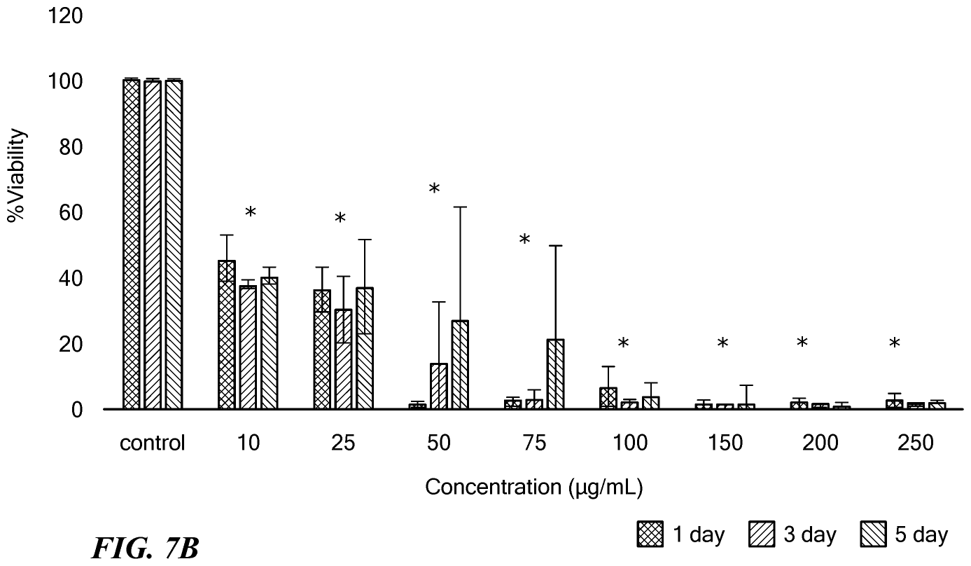

Effectiveness of AgPt nanoparticles as anticancer agent was tested using melanoma and glioblastoma cells and the above-described cytotoxicity assay. The results obtained over all the concentrations tested for melanoma and glioblastoma cells are presented in FIGS. 7A and 7B, respectively. A high percentage of death was observed in melanoma cells due to the treatment with nanoparticles (FIG. 7A). For the three concentrations that showed limited toxicity toward fibroblasts (10, 25, 50 μg/mL), the nanoparticles demonstrated a remarkable and statistically significant capability to kill cancer cells. The results obtained with glioblastoma cells were similar. The effect of the nanoparticles was higher on glioblastoma cells which showed a cell viability of about 40% compared to controls (FIG. 7B). The IC₅₀ obtained for AgPt nanoparticles against melanoma and glioblastoma cells was about 50 μg/mL for all of the days measured.

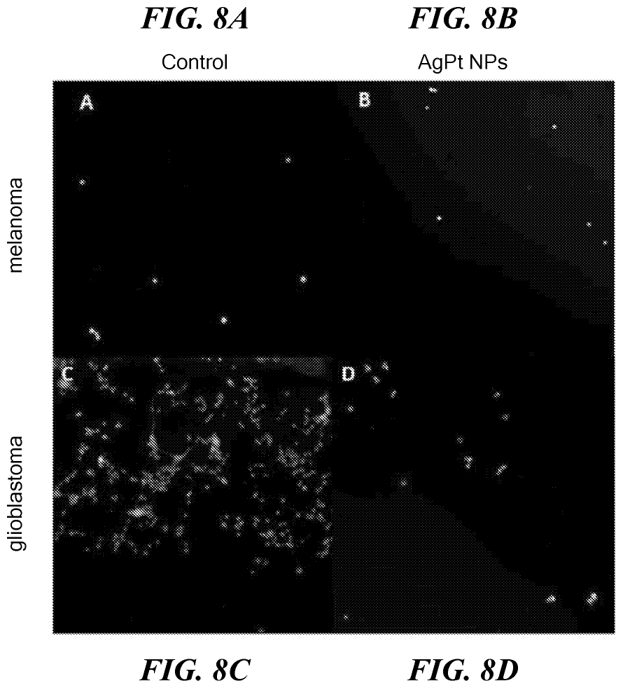

Live/dead assays: A fluorescence microscopy based live/dead assay for cells, like the one described above for bacteria, was performed to obtain a qualitative measurement of the effect of nanoparticles on cells. Cells (melanoma or glioblastoma cells; 50,000/mL) were incubated in a 96-well plate with or without AgPt nanoparticles for 24 hours. After incubation, the plate was centrifuged to remove the supernatant and 100 μl of 0.85% NaCl was added (two times). Subsequently, 1.25 μL of each fluorescent solution from the BacLight™ bacterial viability kit (Thermo Fisher Scientific), in 1 mL of a 0.85% NaCl solution, was added to each well, and the mixture maintained in dark for 15 min. Next, the solution was centrifuged, washed with 0.85% NaCl, and resuspended for fluorescence microscopy (Zeiss inverted fluorescence microscope SpectraMax). Results showed again that the anticancer activity of the nanoparticles was much higher than that of control samples (FIG. 8). Images of glioblastoma cells treated with AgPt nanoparticles included more dead cells; the total number of cells was lower. This indicates that the nanoparticles not only killed cancer cells, but also prevented cell growth.

### REFERENCES

1. Mi, G., Shi, D., Herchek, W. & Webster, T. J. Self-assembled arginine-rich peptides as effective antimicrobial agents. J. Biomed. Mater. Res. Part A 105, 1046-1054 (2017).
2. Ventola, C. L. The antibiotic resistance crisis: part 1: causes and threats. P T 40, 277-83 (2015).
3. Harris, M. Pharmaceutical Microbiology. Academic Medicine 39, xix (1964).
4. Antibiotic/Antimicrobial Resistance CDC. at <https://www.cdc.gov/drugresistance/>
5. Peleg, A. Y. & Hooper, D. C. Hospital-acquired infections due to gram-negative bacteria. N Engl. J. Med. 362, 1804-13 (2010).
6. Tenover, F. C. Mechanisms of Antimicrobial Resistance in Bacteria. Am. J. Med. 119, S3-S10 (2006).
7. Golkar, Z., Bagasra, O. & Pace, D. G. Bacteriophage therapy: a potential solution for the antibiotic resistance crisis. J. Infect. Dev. Ctries. 8, (2014).
8. Marshall, B. M. & Levy, S. B. Food animals and antimicrobials: impacts on human health. Clin. Microbiol. Rev. 24, 718-33 (2011).
9. Fair, R. J. & Tor, Y. Antibiotics and bacterial resistance in the 21st century. Perspect. Medicin. Chem. 6, 25-64 (2014).
10. Livermore, D. M. Bacterial Resistance: Origins, Epidemiology, and Impact. Clin. Infect. Dis. 36, S11-S23 (2003).
11. Nikaido, H. Multidrug resistance in bacteria. Annu. Rev. Biochem. 78, 119-46 (2009).
12. Chatterjee, T., Chatterjee, B. K., Majumdar, D. & Chakrabarti, P. Antibacterial effect of silver nanoparticles and the modeling of bacterial growth kinetics using a modified Gompertz model. Biochim. Biophys. Acta 1850, 299-306 (2014).
13. MacDonald, V. Chemotherapy: managing side effects and safe handling. Can. Vet. J.=La Rev. Vet. Can. 50, 665-8 (2009).
14. Jabir, N. R. et al. Nanotechnology-based approaches in anticancer research. Int. J. Nanomedicine 7, 4391-408 (2012).
15. Taylor, E. & Webster, T. J. Reducing infections through nanotechnology and nanoparticles. Int. J. Nanomedicine 6, 1463-73 (2011).
16. Jayawardena, H. S. N., Jayawardana, K. W., Chen, X. & Yan, M. (Supplemental) Maltoheptaose promotes nanoparticle internalization by Escherichia coli. Chem. Commun. (Camb). 49, 3034-6 (2013).
17. Chen, W.-Y. et al. Self-Assembly of Antimicrobial Peptides on Gold Nanodots: Against Multidrug-Resistant Bacteria and Wound-Healing Application. Adv. Funct. Mater. 25, 7189-7199 (2015).
18. Ishihara, M., Nguyen, V. Q., Mori, Y., Nakamura, S. & Hattori, H. Adsorption of silver nanoparticles onto different surface structures of chitin/chitosan and correlations with antimicrobial activities. Int. J. Mol. Sci. 16, 13973-13988 (2015).
19. Jahromi, E. Z. et al. Palladium complexes: new candidates for anti-cancer drugs. J. Iran. Chem. Soc. 13, 967-989 (2016).
20. Bragg, P. D. & Rainnie, D. J. The effect of silver ions on the respiratory chain of Escherichia coli. Can. J. Microbiol. 20, 883-9 (1974).
21. Li, W.-R. et al. Antibacterial activity and mechanism of silver nanoparticles on Escherichia coli. Appl. Microbiol. Biotechnol. 85, 1115-1122 (2010).
22. Reedijk, J. The mechanism of action of platinum anti-tumor drugs. Pure &Appl. Chem 59, 181-192 (1987).
23. Wang, D. & Lippard, S. J. Cellular processing of platinum anticancer drugs. Nat. Rev. Drug Discov. 4, 307-320 (2005).
24. Fuertes, M. a., Alonso, C. & Perez, J. M. Biochemical modulation of cisplatin mechanisms of action: Enhancement of antitumor activity and circumvention of drug resistance. Chem. Rev. 103, 645-662 (2003).
25. Botchway, S. W. et al. Time-resolved and two-photon emission imaging microscopy of live cells with inert platinum complexes. Proc. Natl. Acad. Sci. U S. A. 105, 16071-6 (2008).
26. Shim, K. et al. Synthesis and Cytotoxicity of Dendritic Platinum Nanoparticles with HEK-293 Cells. Chem. —An Asian J. 12, 21-26 (2017).
27. Hanaor, D., Michelazzi, M., Leonelli, C. & Sorrell, C. C. The effects of carboxylic acids on the aqueous dispersion and electrophoretic deposition of ZrO2. J. Eur. Ceram. Soc. 32, 235-244 (2012).

As used herein, “consisting essentially of” allows the inclusion of materials or steps that do not materially affect the basic and novel characteristics of the embodiment. Any recitation herein of the term “comprising”, particularly in a description of components of a composition or in a description of elements of a device, can be exchanged with “consisting essentially of” or “consisting of”.

While the present technology has been described in conjunction with certain preferred embodiments, one of ordinary skill, after reading the foregoing specification, will be able to effect various changes, substitutions of equivalents, and other alterations to the compositions and methods set forth herein.

What is claimed is:

1. A method for treating bacterial infection, the method comprising administering to a subject in need thereof an effective amount of a composition comprising a plurality of nanoparticles, the nanoparticles comprising an alloy of Ag and either Pt or Pd.

2. The method of claim 1, wherein the nanoparticles further comprise a nonionic surfactant.

3. The method of claim 2, wherein the nanoparticles comprise or consist of Ag, Pd, and a nonionic surfactant.

4. The method of claim 2, wherein the nanoparticles comprise or consist of Ag, Pt, and a nonionic surfactant.

5. The method of claim 2, wherein the nonionic surfactant is polyethylene glycol hexadecyl ether.

6. The method of claim 1, wherein the diameter of the nanoparticles is in the range of about 30 to about 60 nm.

7. The method of claim 1, wherein the average diameter of the nanoparticles is less than about 50 nm.

8. The method of claim 1, wherein the administration of said composition has a bacteriocidal effect or a bacteriostatic effect in the subject.

9. The method of claim 8, wherein the bacteriocidal or bacteriostatic effect is operative against Gram-negative, Gram-positive, and/or multidrug resistant bacteria in the subject.

10. A method for treating cancer, the method comprising administering to a subject in need thereof an effective amount of a composition comprising a plurality of nanoparticles, the nanoparticles comprising an alloy of Ag and either Pt or Pd.

11. The method of claim 10, wherein the nanoparticles further comprise a nonionic surfactant.

12. The method of claim 11, wherein the nanoparticles comprise or consist of Ag, Pd, and a nonionic surfactant.

13. The method of claim 11, wherein the nanoparticles comprise or consist of Ag, Pt, and a nonionic surfactant.

14. The method of claim 11, wherein the nonionic surfactant is polyethylene glycol hexadecyl ether.

15. The method of claim 10, wherein the diameter of the nanoparticles is in the range of about 30 to about 60 nm.

16. The method of claim 10, wherein the average diameter of the nanoparticles is less than about 50 nm.

17. The method of claim 10, wherein the administration of said composition inhibits the growth and/or proliferation of cancer cells, or kills cancer cells, in the subject.

18. The method of claim 17, wherein the administration of said composition also has a bacteriostatic or bacteriocidal effect in the subject.

19. The method of claim 10, wherein administration of an anticancer therapeutic agent is reduced or eliminated in the subject as a result of the administration of said composition.

20. A medical device or consumer article comprising a plurality of nanoparticles on an exposed surface thereof, or impregnated within the article, the nanoparticles comprising an alloy of Ag and either Pt or Pd, and the nanoparticles optionally comprising a nonionic surfactant.

21. The medical device or consumer article of claim 20, wherein said nanoparticles form a coating on said exposed surface, and the coating has a bacteriocidal effect or a bacteriostatic effect.

22. The medical device or consumer article of claim 20, wherein the nonionic surfactant is polyethylene glycol hexadecyl ether.

23. The medical device or consumer article of claim 20 which is an implantable medical device.

24. The medical device or consumer article of claim 21 which is a consumer article, wherein the nanoparticles are present on a surface of the consumer article configured for contact with a user.

25. The medical device or consumer article of claim 21 which is selected from the group consisting of a catheter; a wound dressing; a joint replacement device or portion thereof; a pump; an implantable defibrillator or portion thereof; an artificial organ; a surgical screw, tube, rod, pin, or mesh; a biodegradable device; an electrode; a surgical instrument; an eating implement, plate, cup, glass, or straw; a door knob or handle; an item of sports equipment; a shoe or boot liner; an item of clothing; a comb, brush, or razor; a toothbrush, toothpick, dental floss, or dental instrument or implant; a keyboard or computer tracking device; a touch screen of an electronic device; a remote control device; a musical instrument or part thereof; a writing implement; a packaging material for an article of commerce; and a button, control knob, steering wheel, or gear shift lever of a vehicle.

26. A composition comprising a plurality of nanoparticles, the nanoparticles comprising an alloy of Ag and either Pt or Pd, the composition for use in preventing or reducing the growth of bacteria or cancer cells.

27. Use of a composition comprising a plurality of nanoparticles, the nanoparticles comprising an alloy of Ag and either Pt or Pd, to produce a medicament for preventing or reducing the growth of bacteria or cancer cells.
